(19) United States
(12) Patent Application Publication
(10) Pub. No.: US 2015/0025405 A1
(43) Pub. Date: Jan. 22, 2015

(54) ACUTE LUNG INJURY (ALI)/ACUTE RESPIRATORY DISTRESS SYNDROME (ARDS) ASSESSMENT AND MONITORING

(71) Applicant: KONINKLIJKE PHILIPS N.V., EINDHOVEN (NL)

(72) Inventors: Srinivasan Vairavan, Ossining, NY (US); Caitlyn Chiofolo, New Hyde Park, NY (US); Nicolas Chbat, White Plains, NY (US); Monica Ghosh, Chappaqua, NY (US)

(21) Appl. No.: 14/379,376

(22) PCT Filed: Feb. 14, 2013
(86) PCT No.: PCT/IB2013/051201

§ 371 Date: Aug. 18, 2014

Related U.S. Application Data
(60) Provisional application No. 61/762,988, filed on Feb. 11, 2013.
(60) Provisional application No. 61/600,308, filed on Feb. 17, 2012.

(51) Int. Cl. G06F 19/00; A61B 5/00; A61B 5/08

(52) U.S. Cl. CPC G06F 19/34; A61B 5/08; A61B 5/7271
USPC 600/529

(57) ABSTRACT

A patient is monitored for a medical condition such as acute lung injury (AL1) by operations including: (i) receiving values of a plurality of physiological parameters for the patient; (ii) computing an AL1 indicator value based at least on the received values of the plurality of physiological parameters for the patient; and (iii) displaying a representation of the computed AL1 indicator value on a display (14, 22). The computing operation (ii) may employ various inference algorithms trained on a training set comprising reference patients to distinguish between reference patients having AL1 and reference patients not having AL1, or may employ an aggregation of two or more such inference algorithms. If patients in an ICU are monitored, the display (22) may simultaneously display a diagrammatic representation of each patient including an identification of the patient and a representation of the AL1 indicator value for the patient.

The following relates to the medical monitoring arts, clinical decision support system arts, intensive care monitoring and patient assessment arts, and so forth.

Acute lung injury (ALI) is a devastating complication of acute illness and one of the leading causes of multiple organ failure and mortality in the intensive care unit (ICU). ALI is also sometimes known as Acute Respiratory Distress Syndrome (ARDS). ALI is estimated to be prevalent in 7-10% of all ICU patients, and exhibits a high mortality of greater than 40% after hospital discharge. However, less than one-third of ALI patients are detected by ICU physicians.

One approach for detection or prediction of ALI is known as the ALI prediction score, which uses chronic and acute illness information to identify patients who are more likely to develop ALI during their stay. This approach, however, provides little insight into the timing of development. Another known approach is the ALI sniffer, which is an electronic system for surveying patients' electronic medical records for evidence of ALI. The ALI sniffer is highly sensitive and specific. However, it applies the current ALI definition to the medical record, which is defined in terms of arterial blood gas (ABG) and chest radiograph characteristics. Thus, the ALI sniffer is limited by its reliance on availability of ABG analysis and chest x-ray tests for the patient. Obtaining and utilizing radiographic evidence of bi-lateral infiltrates signifying ALI can be resource intensive, time consuming, and deleterious to the patient, and in many ICU cases the relevant data is not available at least during the critical initial stages of patient admission and triage.

The following contemplates improved apparatuses and methods that overcome the aforementioned limitations and others.

According to one aspect, a non-transitory storage medium stores instructions executable by an electronic data processing device including a display to monitor a patient for acute lung injury (ALI) by operations including: (i) receiving values of a plurality of physiological parameters for the patient; (ii) computing an ALI indicator value based at least on the received values of the plurality of physiological parameters for the patient; and (iii) displaying a representation of the computed ALI indicator value on the display.

According to another aspect, an apparatus comprises an electronic data processing device including a display, and a non-transitory storage medium as set forth in the immediately preceding paragraph operatively connected with the electronic data processing device to execute the instructions stored on the non-transitory storage medium to monitor a patient for acute lung injury (ALI).

According to another aspect, a method comprises: receiving values of a plurality of physiological parameters for a patient in an intensive care unit (ICU) at an electronic data processing device including a display; using the electronic data processing device, computing an indicator value for a medical condition (which in some embodiments is ALI) based at least on the received values of the plurality of physiological parameters for the patient using an inference algorithm trained on a training set comprising reference patients to distinguish between reference patients having the medical condition and reference patients not having the medical condition; and displaying a representation of the computed indicator value on the display of the electronic data processing device.

One advantage resides in providing ALI assessment with timely and available data without solely relying upon radiographic data (e.g. x-rays) or laboratory tests (e.g., arterial blood gas, ABG, analysis).

Another advantage resides in providing ALI assessment that takes into account the impact of drugs or medications administered to the patient.

Another advantage resides in providing ALI assessment that is readily integrated with existing patient monitors commonly used in intensive care and triage settings.

Numerous additional advantages and benefits will become apparent to those of ordinary skill in the art upon reading the following detailed description.

The invention may take form in various components and arrangements of components, and in various process operations and arrangements of process operations. The drawings are only for the purpose of illustrating preferred embodiments and are not to be construed as limiting the invention.

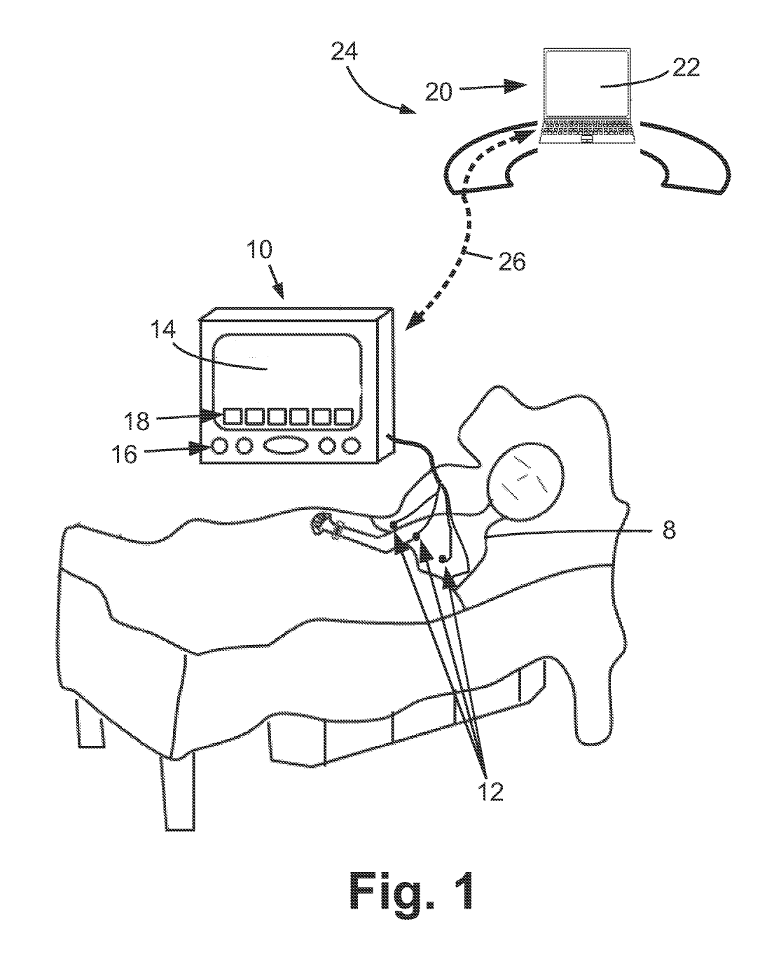

FIG. 1 diagrammatically shows a patient in an intensive care unit (ICU) being monitored for acute lung injury (ALI) at a bedside monitor and at a nurses' station, the latter along with other patients in the ICU.

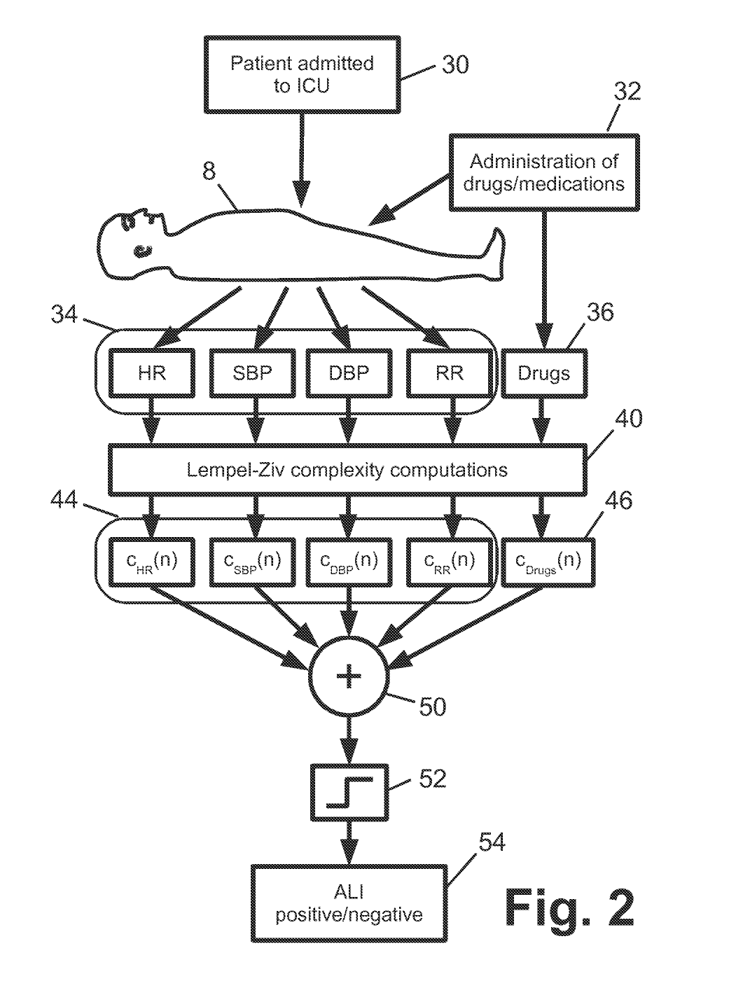

FIGS. 2-4 illustrate an ALI detection approach employing Lempel-Ziv complexity metrics computed for monitored vital signs.

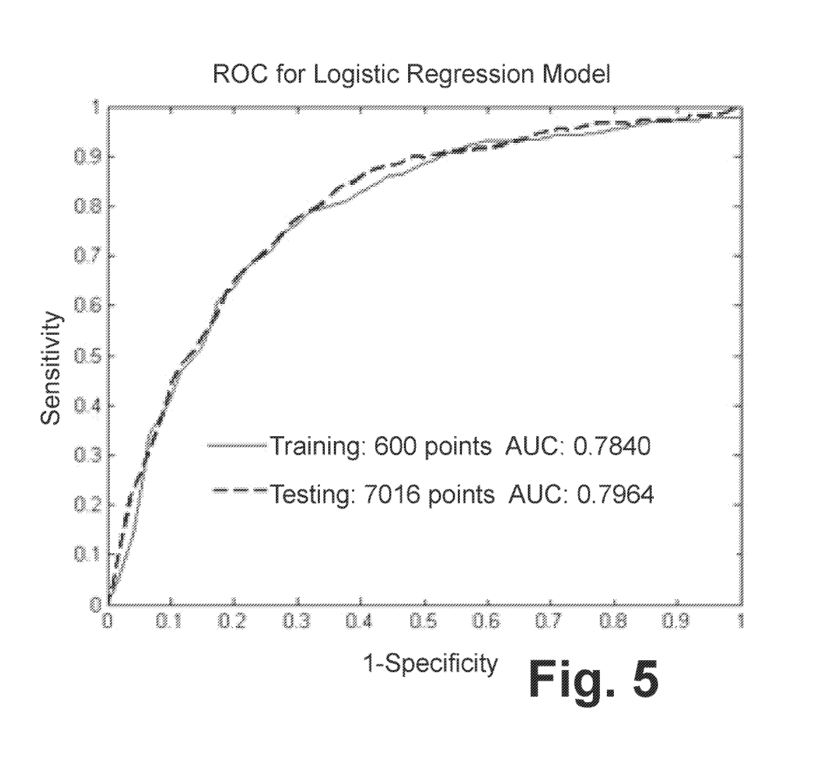

FIG. 5 illustrates experimental results for a logistic regression-based approach for ALI detection.

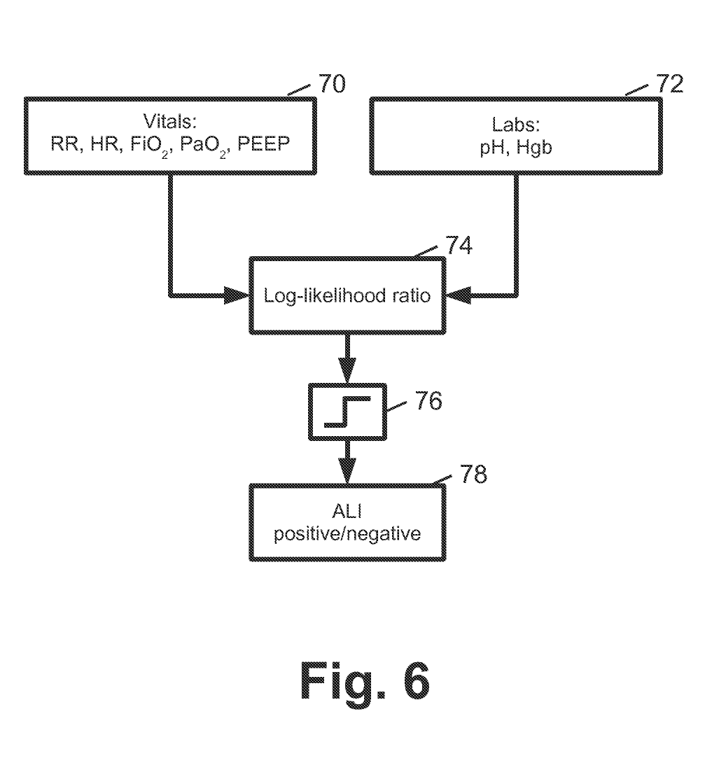

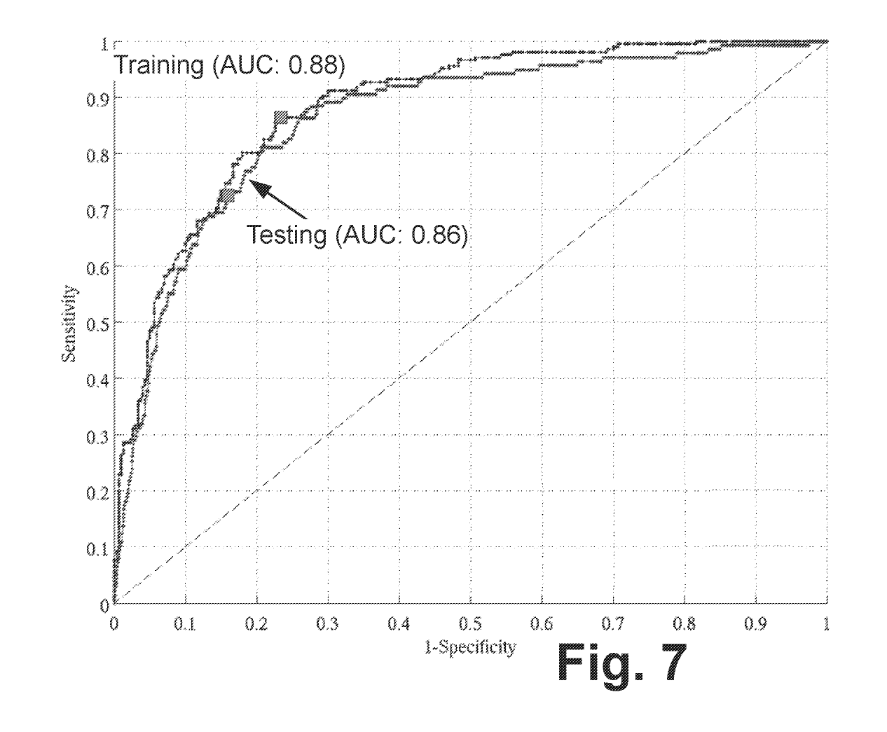

FIGS. 6-7 illustrate a log-likelihood ratio (LLR)-based approach for ALI detection.

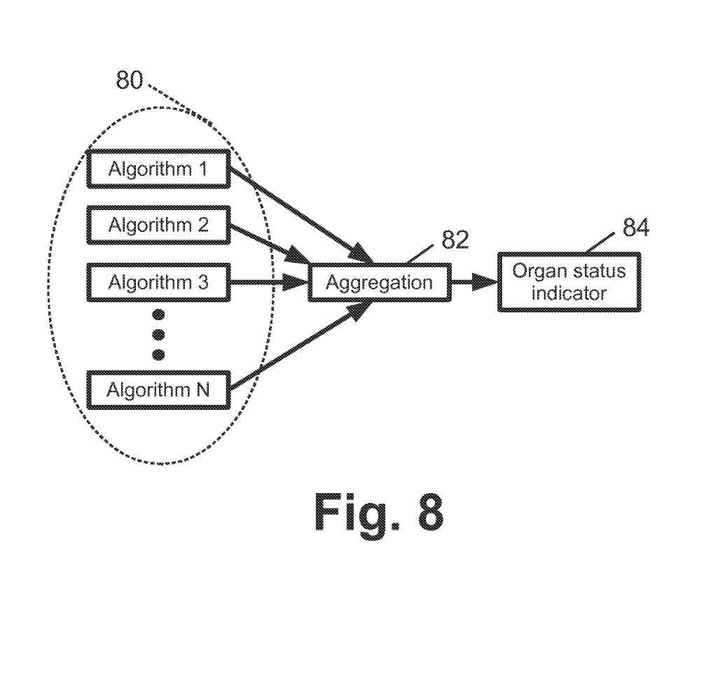

FIG. 8 shows a generic aggregation approach for computing an indicator for a medical condition as an aggregation of constituent indicator algorithms.

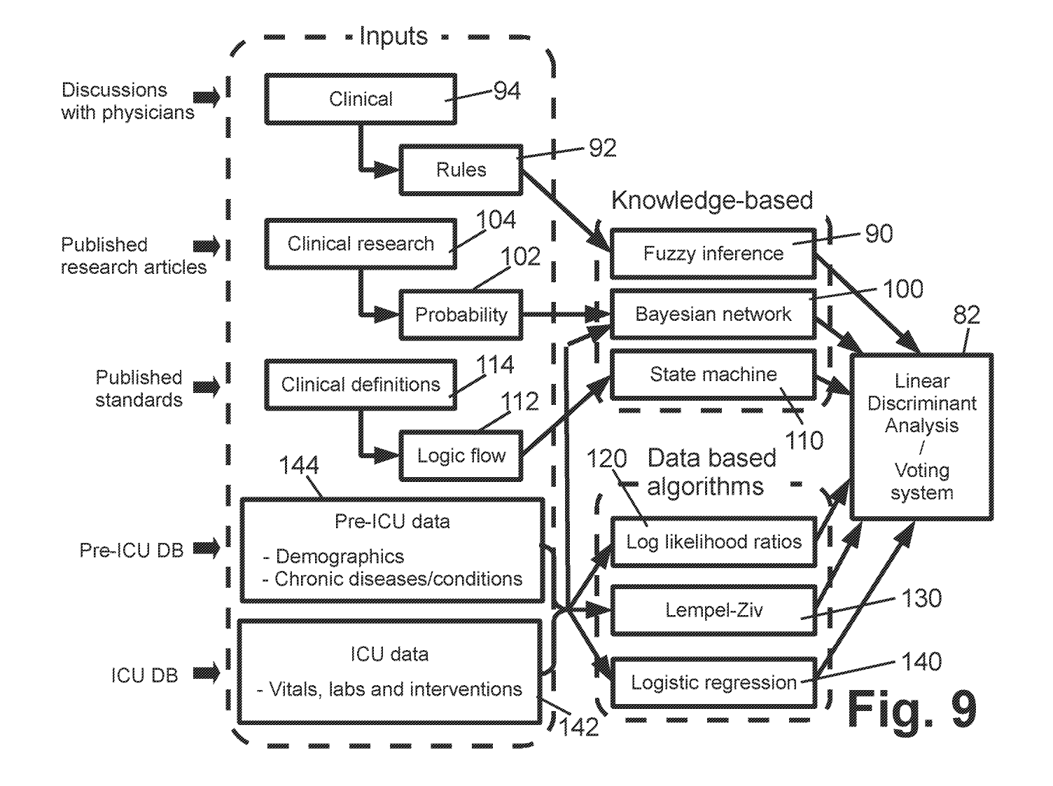

FIGS. 9-15 illustrate application of the aggregation approach of FIG. 8 to a set of constituent ALI indicator algorithms to generate an aggregate ALI indicator.

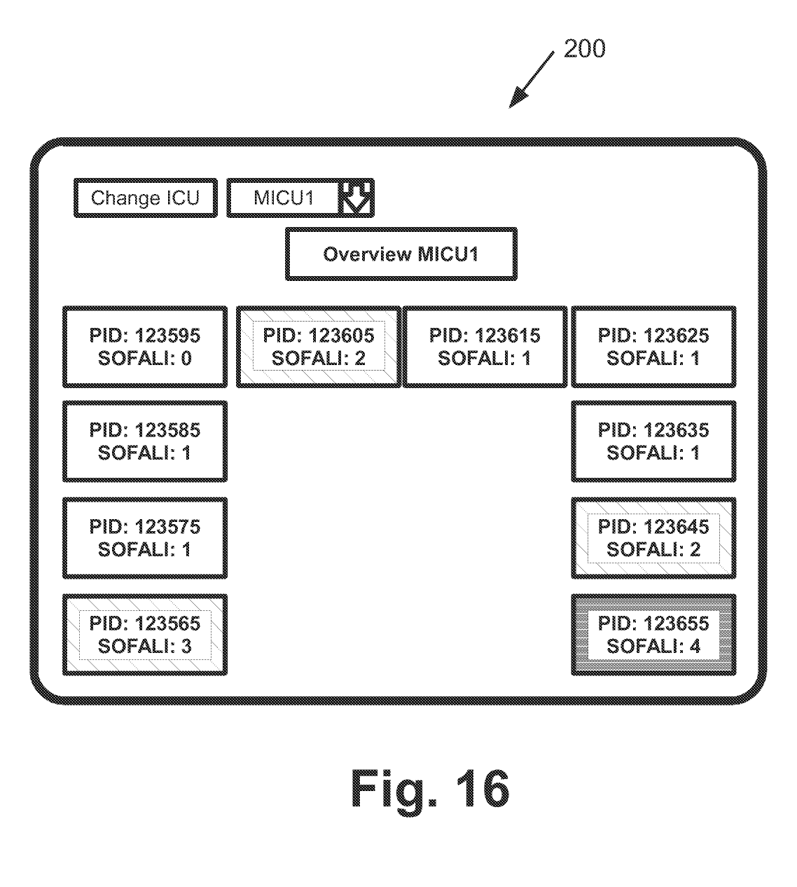

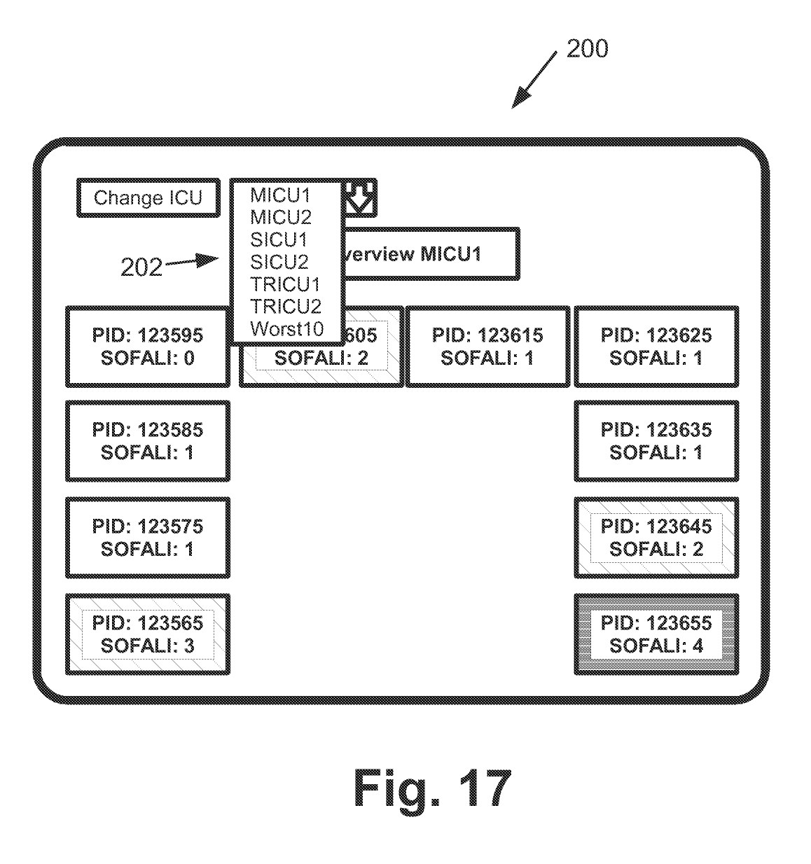

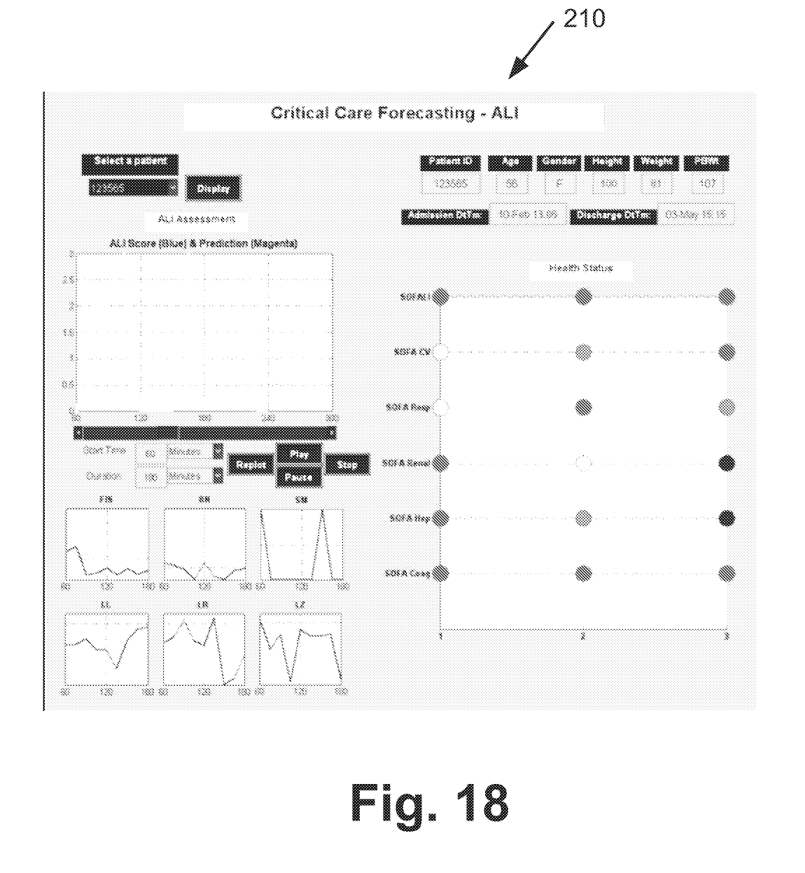

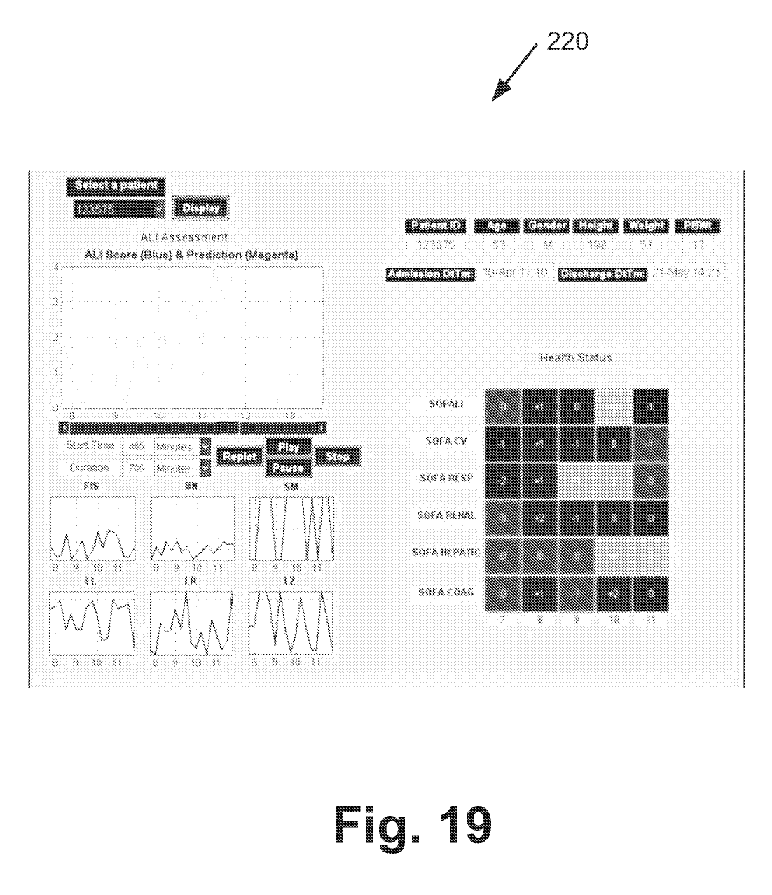

FIGS. 16-19 illustrate displays during various phases of operation of multi-patient monitoring employing an overview display (FIGS. 16-17) and zoom-in displays for a selected patient (FIGS. 18-19).

With reference to FIG. 1, a patient 8 is monitored by a bedside patient monitor 10, which displays trend data for various physiological parameters of the patient 8. (Terms such as “physiological parameters”, “vital signs”, or “vitals” are used interchangeably herein). For example, illustrative electrocardiograph (ECG) electrodes 12 suitably monitor heart rate and optionally full ECG traces as a function of time. Substantially any physiological parameter of medical interest may be monitored, such as by way of illustrative example on or more of the following: heart rate (HR); respiration rate (RR); systolic blood pressure (SBP); diastolic blood pressure (DBP); fraction of inspired oxygen (FiO₂); partial pressure of oxygen in arterial blood (PaO₂); positive end-expiratory pressure (PEEP); blood hemoglobin (Hgb); and so forth.

The patient monitor 10 includes a display 14, which is preferably a graphical display, on which physiological parameters and optionally other patient data are displayed using numeric representations, graphical representations, trend lines, or so forth. The patient monitor 10 further includes one or more user input devices, such as illustrative controls 16 mounted on the body of the monitor 10, a set of soft keys 18 shown on the display 14 (which is suitably a touch-sensitive display in such a configuration), a pull-out keyboard, various combinations thereof, or so forth. The user input device(s) enable a nurse or other medical person to configure the monitor 10 (e.g. to select the physiological parameters or other patient data to be monitored and/or displayed), to set alarm settings, or so forth. Although not explicitly shown, the patient monitor 10 may include other features such as a speaker for outputting an audio alarm if appropriate, one or more LEDs or lamps of other types to output visual alarms, and so forth.

The patient monitor 10 is an “intelligent” monitor in that it includes or is operatively connected with data processing capability provided by a microprocessor, microcontroller, or the like connected with suitable memory and other ancillary electronics (details not illustrated). In some embodiments the patient monitor 10 includes internal data processing capability in the form of a built-in computer, microprocessor, or so forth, such that the patient monitor can perform autonomous processing of monitored patient data. In other embodiments the patient monitor is a “dumb terminal” that is connected with a server or other computer or data processing device that performs the processing of patient data. It is also contemplated for a portion of the data processing capability to be distributed amongst intercommunicating body-worn sensors or devices mounted on the patient 8, e.g. in the form of a Medical Body Area Network (MBAN).

In illustrative examples, the patient 8 is disposed in a patient room of an intensive care unit (ICU), which may for example be a medical ICU (MICU), a surgical ICU (SICU), a cardiac care unit (CCU), a triage ICU (TRICU), or so forth. In such settings, the patient is typically monitored by the bedside patient monitor 10 located with the patient (e.g., in the patient's hospital room) and also by an electronic monitoring device 20 with suitable display 22 (e.g. a dedicated monitor device or a suitably configured computer) located at a nurses' station 24. Typically, the ICU has one or more such nurses' stations, with each nurses' station assigned to a specific set of patients (which may be as few as a single patient in extreme situations). A wired or wireless communication link (indicated diagrammatically by double-arrow-headed curved line 26) conveys patient data acquired by the bedside patient monitor 10 to the electronic monitoring device 20 at the nurses' station 24. The communication link 26 may, for example, comprise a wired or wireless Ethernet (dedicated or part of a hospital network), a Bluetooth connection, or so forth. It is contemplated for the communication link 26 to be a two-way link i.e., data also may be transferrable from the nurses' station 24 to the bedside monitor 10.

The bedside patient monitor 10 is configured to detect and indicate Acute Lung Injury (ALI) by performing data processing as disclosed herein on information including at least one or more physiological parameters monitored by the patient monitor 10. Additionally or alternatively, the electronic monitoring device 20 at the nurses' station 24 may be configured to detect and indicate ALI by performing data processing as disclosed herein on information including at least one or more physiological parameters monitored by the patient monitor 10. Note that the terms ALI and Acute Respiratory Distress Syndrome (ARDS) are used interchangeably herein. Advantageously, the ALI detection as disclosed herein is based on physiological parameters such as HR, RR, SBP, DBP, FiO₂, PEEP, or so forth, which are monitored by the patient monitor 10 and hence are available in real-time. Patient data with longer acquisition latency times, such as radiography reports and laboratory findings (e.g. PaO₂, Hgb, et cetera) are not utilized or are utilized as supplemental information for evaluating whether ALI is indicated.

In the following, various embodiments of ALI/ARDS detection are set forth.

With reference to FIGS. 2-4, an embodiment employing Lempel-Ziv complexity-based detection of ALI is described. Referencing diagrammatic FIG. 2, the patient 8 is admitted to the ICU (indicated by block 30). There may be scenarios where different drugs/medications (“drugs” and “medications” are used interchangeably herein) may be administered to the patient 8 in order to stabilize the patient (indicated by block 32). The illustrative ALI detection approach of FIG. 2 utilizes illustrative vital signs data streams 34 including heart rate (HR), arterial systolic and diastolic blood pressure (SBP and DBP), and respiratory rate (RR), along with an additional patient data stream 36 comprising instances of the administration 32 of one or more different drugs to the patient 8. The drug administration data stream 36 can take various forms, such as a binary data stream (e.g. value “0” as a function of (optionally discretized) time except during a drug administration event which is indicated by a value “1”. In the case of a drug administered over a time interval, e.g. an intravenous drip, the value may be “0” when no drip is being administered and “1” (or some other value) during the administration of the drip. Other value-time representations are also contemplated, e.g. a time-varying value modeling the expected dynamic drug concentration in the patient (or in an organ of interest) from initial administration until the drug is removed from the body by the kidneys or other mechanism.

In a block 40, the Lempel-Ziv complexity metric (see e.g. A. Lempel and J. Ziv, “On the complexity of finite sequences,” IEEE Trans. Inform. Theory, vol. IT-22, pp. 75-81, 1976) is computed for each of the vital sign data streams 34 and for the drug administration data stream 36. This generates a Lempel-Ziv complexity metric 44 corresponding to each vital sign data stream 34, and a Lempel-Ziv complexity metric 46 corresponding to the drug administration data stream 36. The Lempel-Ziv complexity metrics 44, 46 are combined by an addition 50 (optionally with weighting of the data streams) or by another aggregation operator to generate an additive complexity value that is then thresholded by a thresholder 52 to generate a binary ALI indicator 54 having a positive (or other designated) value indicating the patient exhibits ALI or a negative (or other designated) value indicating the patient does not exhibit ALI.

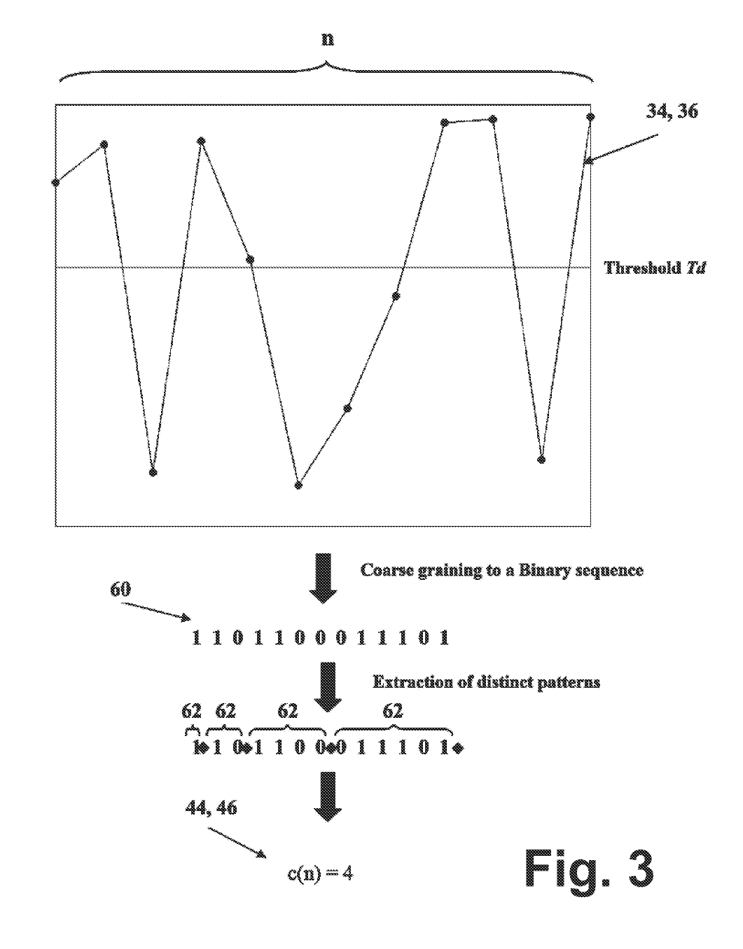

With reference to FIG. 3, operation of the Lempel-Ziv complexity metric computation block 40 is further described. Lempel-Ziv complexity is used to quantify the complexity of different time series signals such as electroencephalography (EEG), heart rate, blood pressure, and so forth. In the system of FIG. 2, the input is a vital sign data stream 34 or the drug administration data stream 36. Lempel-Ziv (LZ) complexity is based on coarse-graining the data stream, i.e. discretizing the data stream in the time (if not already acquired as discrete samples) and value dimensions. In illustrative FIG. 3, the data stream is assumed to already be acquired as discrete time samples, and the value is coarse-grained by converting the numerical data into binary values, e.g “0” if the value is below a threshold T_{d} or “1” if the value is above the threshold T_{d}. Other coarsening approaches are contemplated, e.g. discretizing to a more granular sequence (0, 1, 2, . . . , N) using multiple thresholds. The output of this operation is the coarse-grained, e.g binary, data stream 60.

The LZ complexity is a measure of the amount of distinct patterns available in the sequence, or more particularly within a time interval or time window n of the sequence. In order to obtain the LZ complexity, the binary sequence 60 is scanned from left to right over the window n and a complexity counter is incremented by one unit every time a new (sub-)sequence of consecutive characters is encountered. In the illustrative example of FIG. 3, four sub-sequences 62 are identified in the window n, and thus the Lempel-Ziv complexity measure 44, 46 is in this case c(n)=4. Optionally, some normalization may be applied, e.g. so that the Lempel-Ziv complexity measure c(n) is expressed in units of new pattern occurrences per unit time. It will be appreciated that the processing shown diagrammatically in FIG. 3 may be repeated for successive (and optionally partially overlapping) time windows n to provide the Lempel-Ziv complexity measure c(n) as a function of (discretized) time.

With reference back to FIG. 2, and using the notation employed in FIG. 3, the adder 50 is suitably c_{HR}(n)+c_{SBP}(n)+c_{DBP} (n)+c_{RR}(n)+c_{Drugs}(n). Alternatively, if weighting is employed the output may be written as w_{HR}c_{HR}(n)+w_{SBP}c_{SBP}(n)+w_{DBP}c_{DBP}(n)+w_{RR}c_{RR}(n)+w_{Drugs}c_{Drugs}(n) where the w terms are scalar weights.

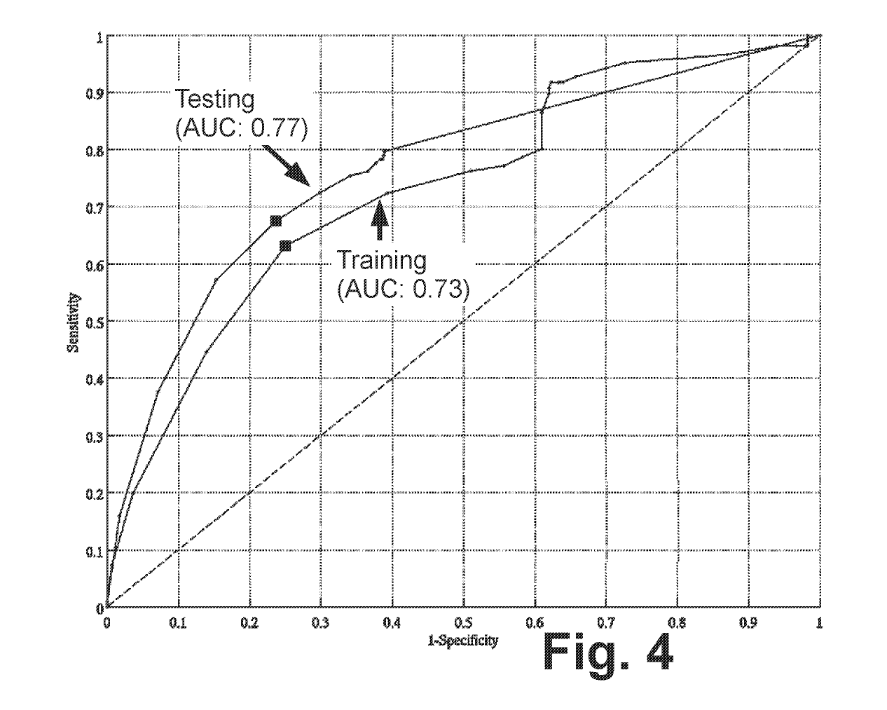

A Receiver Operating Characteristics (ROC) analysis is suitably used in order to obtain the optimal threshold T_{d} of detection for use in the Lempel-Ziv (LZ) complexity measure computation of FIG. 3. In an actually-performed example, ROC analysis for LZ was performed on 506 ICU patients (training datasets), of which 206 where ALI-positive (i.e. exhibited ALI) and 300 were controls (i.e. ALI-negative, did not exhibit ALI). FIG. 4 shows the results for the training population, where the area under the ROC curve is 0.73 and the optimal threshold is 5.92 (sensitivity: 63% and specificity: 75%). The optimal threshold is marked by a black square in FIG. 4. To validate the approach, an ROC analysis was then performed on 6881 ICU patients (unseen test data). Out of these, 138 were ALI-positive and 6743 were controls. The threshold of 5.92 obtained with the training population was located in the ROC curve of testing datasets (also plotted in FIG. 4). The proposed approach achieved a better sensitivity (67%) and better specificity (76%) in the testing datasets. In these actually-performed examples, the summation 50 was unweighted (or, equivalently, all weights were w=1). If nonzero weights are to be employed, they can also be optimized during the training process.

With reference to FIG. 5, an embodiment employing logistic regression-based detection of ALI is described. This illustrative approach entails selecting the features of exploration, fitting a model to a training or derivation dataset of ICU patient data, and testing a model on a validation dataset, preferably one that reflects the true prevalence of ALI in the ICU population of interest.

The logistic regression model involves a nonlinear mapping of the independent or predictor variables such as heart rate (HR), respiratory rate (RR), non-invasive blood pressure measurement (NIBP-m), or so forth, to the dependent or response variable (e.g. ALI or control in the illustrative examples) through the logistic regression function or logit transformation. A suitable formulation is

$$p = \frac{{e}^{{β}_{0} + {β}_{1} {x}_{1} + … {β}_{i} {x}_{i}}}{1 + {e}^{{β}_{0} + {β}_{1} {x}_{1} + … {β}_{i} {x}_{i}}}$$

where p denotes the probability of ALI, β₀ is a constant, and β₁ . . . βᵢ are coefficients of the predictors x₁ . . . xᵢ (e.g., the HR, RR, NIBP-m, et cetera). In a suitable approach, the logistic regression model is fit using the likelihood function L (\vec{β}, β₀)=Πᵢ₌₁ⁿp(\vec{x}ᵢ)^{y}^{₁}(1−p(\vec{x}ᵢ))^{1-y}^{ᵢ} where β₀ is again a constant, \vec{β} is a vector of the coefficients of the predictors, p is again probability of ALI, and y is the true presence/absence of ALI. The coefficients are computed using minimization techniques such as the ordinary least squares (OLS) or the maximum likelihood estimator (MLE).

In an actually performed example, the logistic regression model used three features as input: HR, RR, and HR/NIBP-m, to yield a probability of ALI development. In the training phase, the constant β₀ and coefficients \vec{β} were derived from a 600 patient dataset comprising 300 controls and 300 ALI patients using the foregoing equations. The model was applied continuously (in other words, applied to each unique time point for a patient) and a receiver operator characteristic (ROC) curve was drawn to determine the threshold providing the desired level of sensitivity and specificity. In the testing phase, the model was then applied in the same continuous manner to a validation set of unseen patient data comprising 6,690 controls and 326 ALI patients. An ROC curve was again drawn and the sensitivity and specificity at the previously determined threshold were compared to those obtained from the derivation dataset.

FIG. 5 shows the results. Performance of the logistic regression model on the training data resulted in 71.00% sensitivity and 74.33% specificity. Using the same threshold, performance of the model on the validation data resulted in 63.19% sensitivity and 81.05% specificity.

The actually performed example is merely illustrative. In general, higher or lower frequency data may be employed in the training, testing, and implementation of the logistic regression model. Other embodiments optionally include additional features, such as demographic and baseline health information, to the extent that such data is available via electronic medical records (EMRs) or other sources.

With reference to FIGS. 6 and 7, an embodiment employing log-likelihood ratio (LLR)-based detection of ALI is described. With particular reference to FIG. 6, a flowchart of a suitable log-likelihood ratio based detection of ALI is shown. Let N be the total number of patients in a derivation (i.e. training) data set, of which N₁ have the disease (ALI in the illustrative example) and N₀ do not have the disease. The disease state is denoted as D, i.e D=1 denotes ALI positive and D=0 denotes absence of ALI (i.e. ALI-negative). Let d=[d₁ d₂ . . . d_{L}] denote a vector of patient data that is available to make a diagnosis. In illustrative FIG. 6 these L parameters include vital signs 70, e.g. RR, HR, FiO₂ (fraction of inspired oxygen), PaO₂ (partial pressure of oxygen in arterial blood), PEEP (positive end-expiratory pressure), or so forth, and laboratory test results 72, e.g. pH, Hgb (hemoglobin blood test result), or so forth. As another example (not illustrated), the L parameters may additionally or alternatively include data on whether the patient has one or more acute or chronic conditions such as pneumonia, diabetes, or so forth. The log-likelihood ratio is then defined as

$$L L R ( \underline{d} ) = log [ \frac{p ( \underline{d} / D = 1 )}{p ( \underline{d} / D = 0 )} ]$$

where p(d/D=1) is the joint probability distribution function of d given D=1 and p(d/D=0) is the joint probability distribution function of d given D=0. With the assumption that the L parameters are independent, the log-likelihood ratio can be rewritten as follows:

$$\begin{matrix}L L R ( \underline{d} ) = log [ \frac{{\prod }_{i = 1}^{L} p ( {d}_{i} / D = 1 )}{{\prod }_{i = 1}^{L} p ( {d}_{i} / D = 0 )} ] \\ = {\sum }_{i = 1}^{L} log [ \frac{p ( {d}_{i} / D = 1 )}{p ( {d}_{i} / D = 0 )} ] \\ = {\sum }_{i = 1}^{L} L L R ( {d}_{i} )\end{matrix}$$

Thus, the joint log-likelihood ratio of all the parameters is the sum of the log-likelihood of the individual parameters.

FIG. 6 shows the testing phase. The log-likelihood ratio LLR(d) is computed in an operation 74 for a patient with input patient data vector d whose elements [d₁ d₂ . . . d_{L}] store patient data for the patient under test. The ALI detection then proceeds using a threshold operation 76 as follows:

$$L L R ( \underline{d} ) {≷}_{D = 0}^{D = 1} T$$

That is, if LLR(d)>T then the test result 78 is deemed ALI positive (D=1), whereas if LLR(d)<T then the test result 78 is deemed ALI negative (D=0). In these expressions, T is an optimum detection threshold determined from the training data set.

With reference to FIG. 7, results for an actually performed log-likelihood ratio-based ALI test are reported. An ROC analysis is used in order to obtain the optimal threshold T for the threshold operation 76. ROC analysis for LLR was performed on 506 ICU patients (training dataset), of which 206 where ALI and 300 were controls. The results of the training population are shown in FIG. 7. The area under the ROC curve is 0.88 and the optimal threshold is 2.6 (sensitivity: 86% and specificity: 77%). As more data sets are obtained for training the thresholds and performance values may change. The optimal threshold is marked as a black square in the plot. To validate the approach, an ROC analysis on 6881 ICU patients (unseen test data) was performed. Out of these, 138 were ALI and 6743 were controls. The threshold obtained from the training data is also shown in FIG. 7 in its corresponding location on the ROC curve generated from testing data. The approach achieved a specificity (84%) and sensitivity (72%) in the testing datasets. Location of the operating point (training threshold T) changed slightly in the testing datasets, with decreased sensitivity and increased specificity. However, the threshold is fairly robust considering the increased specificity. The approach also has an area under the ROC curve (0.86) for testing datasets very close to that of the training datasets (0.87) which is advantageous for reliable ALI detection.

The ALI/ARDS detection approaches employing a Lempel-Ziv complexity metric (LZ, described with reference to FIGS. 2-4), a logistic regression-based approach (LR, described with reference to FIG. 5), and a log-likelihood ratio-based approach (LLR, described with reference to FIG. 7) are illustrative examples, and other inference algorithms are contemplated. Such inference algorithms could include a fuzzy inference system, a Bayesian network, and a finite state machine, among others.

With reference to FIGS. 8-15, it is also contemplated to employ various aggregations of inference algorithms, and optionally other information, in detecting (i.e. inferring) the presence of ALI in a patient. The aggregation of such techniques leverages the observation made herein that each algorithm recognizes patterns in the data differently, so that an integrative (e.g. aggregative) approach using complementary information from various unique algorithms in combination is expected to give better performance than any one of the individual algorithms acting alone.

With particular reference to FIG. 8, a generic framework of the integrative approach is disclosed. The outputs of set of N algorithms 80, referred to herein without loss of generality as Algorithm 1, Algorithm 2, Algorithm 3, . . . , Algorithm N, are aggregated at an aggregation block 82 to generate an organ status indicator 84 that is suitably displayed and/or trended as a function of time on the bedside monitor 10, nurses' station monitoring device 20, (see FIG. 1) or so forth. The generic framework of FIG. 8 is not disease-specific.

With reference to FIG. 9, an application of the generic aggregation framework of FIG. 8 to ALI detection is shown. In this application the N algorithms 80 include six algorithms (i.e. N=6) as outlined in the following.

A first algorithm is based on a distillation of physicians' expertise. In illustrative FIG. 9, this is implemented as a fuzzy inference algorithm 90 that is built from linguistic (or fuzzy) information about relationships of variables and run using a set of decision rules 92 constructed based on clinical information 94 collected in discussions with physicians. The fuzzy inference algorithm 90 may, for example, constitute a clinical decision support system (CDSS) component.

A second algorithm is based on distillation of relevant clinical literature. In illustrative FIG. 9, this is implemented as a Bayesian network 100 that is structured from probabilities 102 computed based on clinical research 104. For example, a clinical study may indicate that statistically a combination of parameters is indicative of ALI with a probability P.

A third algorithm is based on the translation of pathophysiology in terms of causal relationships between variables (such as RR, HR, etc.). Potential causes of ALI development could be mechanical, chemical, or biological in nature. For instance, mechanical causes of ALI include fast/deep breathing and/or ventilation settings. Examples of mechanical conditions are:

Ventilation setting of positive end expiratory pressure (PEEP)<5 Condition 1:

PEEP>10 Condition 2:

plateau pressure>35 cmH₂O. Condition 3:

In illustrative FIG. 9, this is implemented as a state machine 110 implementing a logic flow 112 quantifying a clinical definition 114. In the instant case, if all of Conditions 1, 2, or 3 are not met, then the state machine 110 outputs ALI negative, while if any of the three conditions is met then the state machine 110 outputs ALI positive.

These first three algorithms are knowledge-based, and leverage clinical information, published clinical studies, and clinical definitions, respectively. The fourth, fifth, and sixth algorithms are data-based, and in illustrative FIG. 9 correspond to the LLR algorithm 120, LZ algorithm 130, and LR algorithm 140, respectively, described herein with reference to FIGS. 2-7. These algorithms 120, 130, 140 are based on ICU data 142 such as vitals, labs, and interventions (e.g. drug administration events), and are optionally also based on pre-ICU data 144 such as demographic data and/or known chronic diseases or conditions of the patient. (Note that the term “pre-ICU” indicates that such patient information are typically gathered prior to the patient being admitted to the ICU as part of the admissions procedures; however, the pre-ICU data 144 may in some cases be generated, in whole or in part, after the patient enters the ICU).

The aggregation block 82 may be implemented in various ways. In the illustrative ALI application of FIG. 9, the aggregation block 82 is implemented by linear discriminant analysis (LDA) or by a voting system (SOFALI). These illustrative aggregation approaches are described in turn in the following.

The linear discriminant function for each class k can be represented as:

$${y}_{k} ( x ) = - ( \frac{1}{2} ) {μ}_{k} {C}^{- 1} {μ}_{k}^{T} + log ( {p}_{k} ) + ( {μ}_{k}^{T} {C}^{- 1} ) x$$

where x are predictor variables (e.g., the different ALI detection algorithms), pₖ are the prior probabilities of classes k, and C is the pooled covariance matrix across classes. For the illustrative ALI detection application, the LDA coefficients are obtained for the different predictor variables (i.e., different algorithms) on the training data set. LDA coefficients are then suitably passed through a softmax transformation in order to convert the coefficients to probabilities pₖ according to:

$${p}_{k} = \frac{exp ( {y}_{k} )}{{\sum }_{j = 1}^{k} exp ( {y}_{j} )}$$

The voting system aggregator is suitably implemented as follows. The thresholds of the knowledge-based and data-based approaches are obtained from the training data set. These individual thresholds are then used to obtain a voting system based ALI detection (based on the number of algorithms detecting ALI). TABLE 1 shows the illustrative voting system (SOFALI) employed for integrating the six different algorithms of illustrative FIG. 9.

TABLE 1
Voting system for integrating the
different ALI detection algorithms

| Number of algorithms detecting ALI | Votes (SOFALI) |
|---|---|
| Any one or none | 0 |
| 2 | 1 |
| 3 | 2 |
| 4 | 3 |
| 5 or 6 | 4 |

Other embodiments could include a scale of 0 to 1 where the number of votes is normalized by the total number of algorithms present.

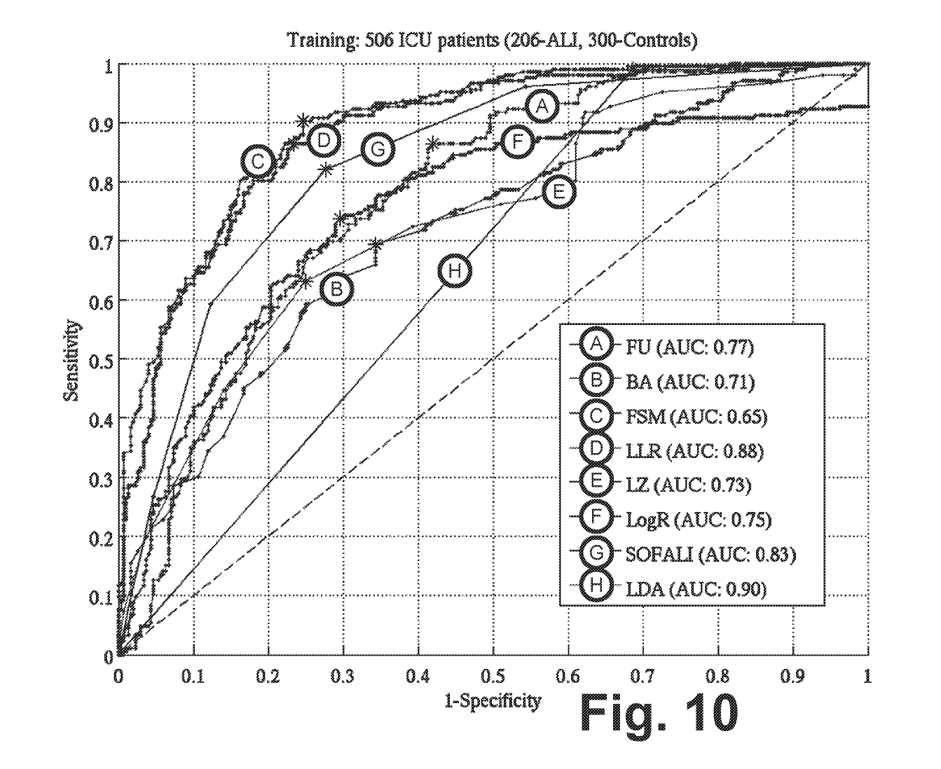

In an actually performed implementation, all of the knowledge-based and data-based and integrative approaches of the illustrative aggregative ALI detection system of FIG. 9 were trained using 506 ICU patient data and validated on an unseen 6881 ICU patient data. Receiver Operating Characteristics curve (ROC) were used to assess the performance of the different approaches. An ROC analysis was used in order to obtain the optimal threshold of ALI detection. ROC analysis for the all different approaches was performed on 506 ICU patients (training datasets), of which 206 where ALI and 300 were controls. The results of the training population are shown in FIG. 10. The optimal threshold for each integrative approach is represented with an asterisk (*) in FIG. 10. The thresholds corresponding to these asterisks are 0.859 for LDA and 2 for SOFALI.

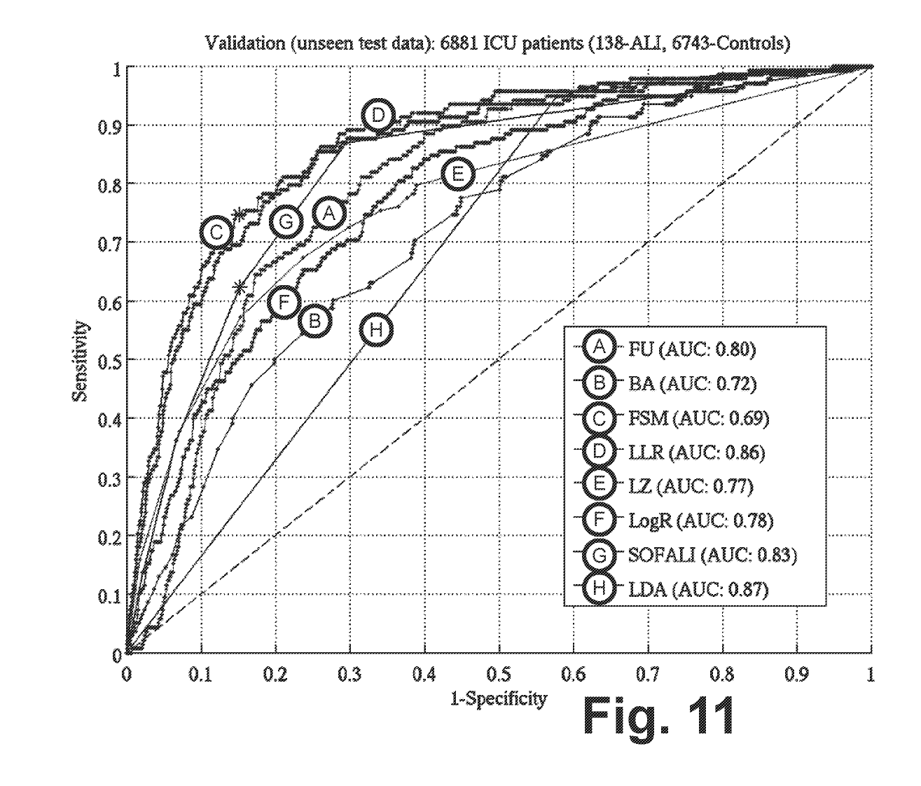

In order to validate the two aggregation approaches, an ROC analysis on 6881 ICU patients (unseen test data) was performed. Out of these, 138 were ALI and 6743 were controls. The thresholds obtained from the training data for LDA and SOFALI respectively and shown in the ROC curve obtained from validation data FIG. 11, change position slightly, with decreased sensitivity and increased specificity, indicating that the threshold is fairly robust. The proposed approaches achieved a better specificity in the testing datasets which is valuable in the context of a reliable ALI detection.

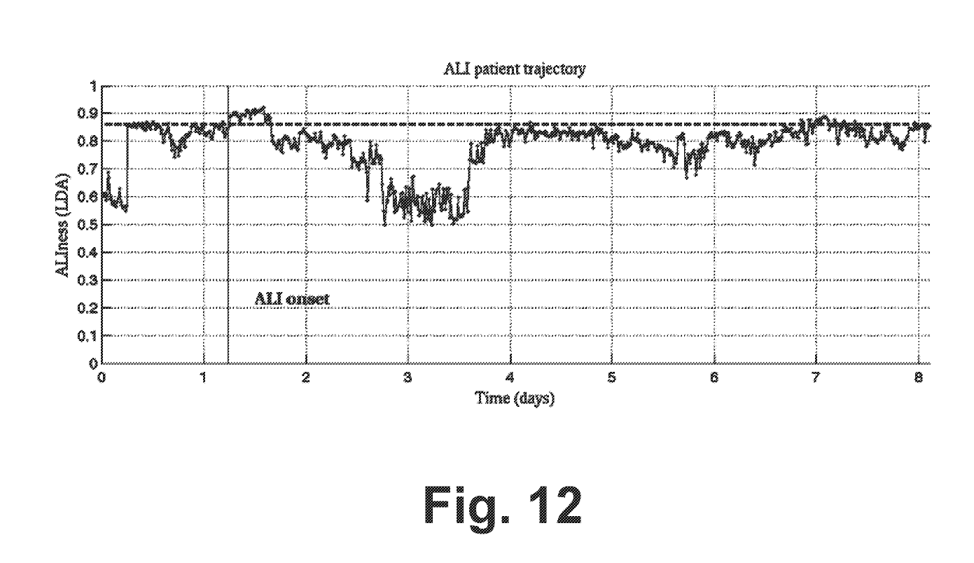

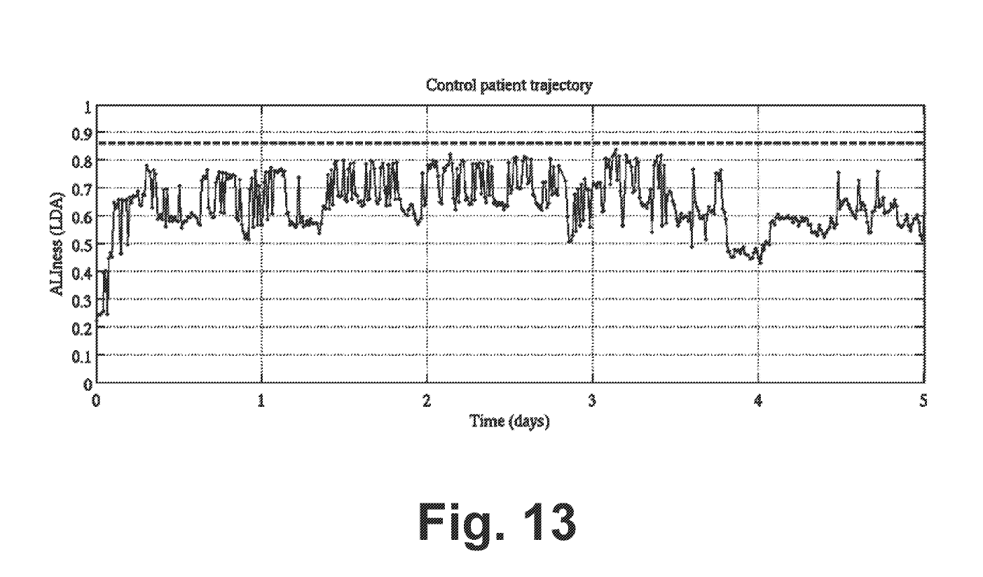

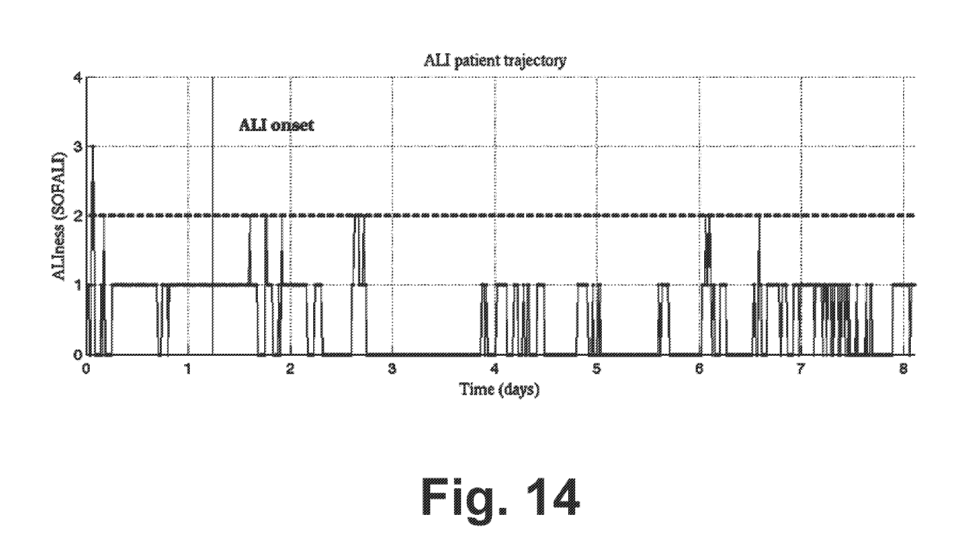

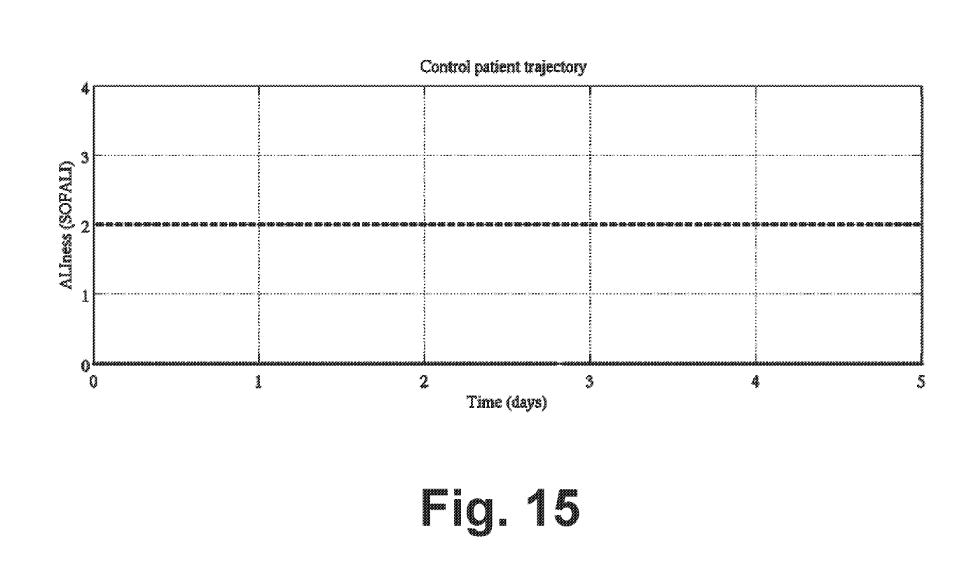

With reference to FIGS. 12 and 13, trajectories of the integrative LDA approach are shown for an illustrative ALI patient (FIG. 12) and for a control patient (FIG. 13). With reference to FIGS. 14 and 15, trajectories of the integrative SOFALI approach are shown for an illustrative ALI patient (FIG. 14) and for a control patient (FIG. 15). FIGS. 12-15 demonstrate that both the LDA and SOFALI integrative approaches detected ALI early as compared to the retrospectively determined ALI onset time by the physician.

The aggregation embodiment described with reference to FIG. 9 is merely illustrative, and numerous variants are contemplated. For example, the set of algorithms can be different from the illustrative six algorithms of FIG. 9. Aggregation algorithms other than LDA or SOFALI are also contemplated, such as aggregation based on a distance metric or based on decision trees or so forth. Moreover, while the illustrative embodiments relate to detection of ALI/ARDS, it will be appreciated that analogous approaches can be employed to detect other illnesses or conditions such as Acute Kidney Injury (AKI), Disseminated Intravascular coagulation (DIC), using suitable vital signs and optionally other features such as the illustrative drug administration data stream, and training on suitable training data sets to optimize the inference algorithm parameters.

The ALI status indicator computed by any of the disclosed algorithms (with or without aggregation) may be utilized in various ways. In the illustrative example, the ALI status indicator may be displayed and optionally logged on the bedside monitor 10 and/or displayed and optionally logged at the nurses' station electronic monitoring device 20 (see FIG. 1). The display can be numeric, and/or in the form of a trend line plotting ALI status indicator value versus time. In the case of an inference engine that generates a value that is thresholded to generate an ALI positive (or negative) indication, it is contemplated to additionally or alternatively display the value without thresholding. For example, the ALI value generated by the inference engine may be plotted as a trend line with the ALI positive/negative threshold shown as a horizontal line superimposed on the trend line graph. Additionally or alternatively, multiple thresholds may be applied to correspond to increasing disease severity or increasing probability of ARDS. Color coding can be applied to indicate the level of severity of the threshold.

Additionally or alternatively, the ALI status indicator can serve as input to a clinical decision support system (CDSS), serving as one piece of data used in conjunction with other data in generating clinical recommendations for consideration by the physician.

In these various applications, the ALI status indicator is typically not accepted as a diagnosis, but rather the ALI status indicator serves as one piece of data for consideration by the patient's physician or other expert medical personnel in deciding the most appropriate course of treatment for the patient.

A typical ICU services several patients at any given time. Each of these patients may (at least in general) be susceptible to ALI/ARDS, and is advantageously monitored for this condition using techniques disclosed herein. However, the ICU is a stressful and complex environment, and additional information such as a set of ALI status indicators for the patients in the ICU may contribute to information overload. In view of this, it is further disclosed herein to provide a multi-patient monitoring display that facilitates rapid review of the condition of all patients in the ICU being monitored for ALI. This multi-patient monitoring display is suitably employed at the nurses' station electronic monitoring device 20 (see FIG. 1) to provide monitoring of all patients under the care of the nurse or nurses (or other medical personnel) assigned to the nurses' station.

With reference to FIG. 16, an illustrative overview multi-patient monitoring display 200 is suitably shown on the nurses' station electronic monitoring device 20 of FIG. 1. The illustrative overview display 200 diagrammatically represents each patient in the current ICU (the medical ICU, i.e. MICU, in illustrative FIG. 16) by a box containing the most pertinent information, in the illustrative example including the patient identification (PID) number and the ALI status indicator value for the patient, represented in illustrative FIG. 16 by the SOFALI aggregation value (more generally, any of the ALI status indicators disclosed herein, with or without aggregation, may be employed). Optionally, the boxes diagrammatically representing the patients are laid out on the display 200 in a manner mimicking the physical layout of the patients in the ICU. In illustrative FIG. 200 the illustrative MICU has ten beds laid out in a “C” pattern and all ten beds are occupied by patients. If a bed was unoccupied, this could be suitably represented by employing an empty box for that bed or by omitting the representative box entirely.

To further facilitate rapid assessment of patient condition, each of the diagrammatic boxes is optionally color-coded to represent the ALI status of the patient. In illustrative FIG. 16, the color coding is diagrammatically represented by different cross-hatchings, with patients having SOFALI index values 0 or 1 being one color (e.g. green or white or no color), patients having SOFALI index values 2 or 3 being a different color (e.g. yellow to indicate a “watch” status for these patients), and patients having SOFALI of 4 (or possibly greater) being yet a different color (e.g. red to indicate a serious ALI or ARDS condition). Alternatively, the color-coding can correspond to severity of illness and a change in color can correspond to a new threshold or boundary of a score ranging. For example, for a score ranging from 0 to 100, 0 to 50 can represent a low risk group, 50 to 75 can indicate a medium risk (“watch” or “warning”) group, and above 75 can indicate a high risk group. With brief reference to FIG. 17, the overview display 200 optionally includes a drop-down menu 202 or other graphical user interface (GUI) dialog enabling a nurse or other operator to switch to a different ICU unit.

The information contained in the diagrammatic boxes of the overview display 200 is merely an illustrative example, and additional or other information may be shown. For example, patients may be identified by name instead of or in addition to by PID number. Other serious conditions may be indicated instead of or in addition to ALI. If two or more conditions are indicated and are to be represented by color coding, the color coding may be shown in different areas of the box, or the entire box may be color coded by the color representing the most serious condition (e.g. “red” if any represented condition has a “red” status color, even if some other displayed condition would be “yellow” or “white”).

In various embodiments, the multi-patient overview display provides a quick “snapshot” overview of critical health status of a group of patients in the ICU, or in other locales (e.g. ED, OR, ward, etc.), via diagrammatic health status blocks. In various embodiments, one or more of the following may be incorporated: (1) individual color-coded block with numeric value and label (e.g. overall health); (2) individual color-coded block with numeric value and label (e.g. ALI health); (3) Multiple color-coded blocks contained within a single block with numeric values and labels (e.g. acute lung injury, acute kidney injury, disseminated intravascular coagulation, acute myocardial infarction, et cetera); or so forth. In general, each diagrammatic block of the overview display provides an overall view of critical illness status of an individual patient, and the collection of blocks in the overview display thus provides this information for all patients in the ICU.

With reference to FIGS. 18 and 19, by selecting the diagrammatic box representing a particular patient, for example by clicking on the box using a mouse or other pointing device, touching the box in the case of a touchscreen, or so forth, a zoomed-in view of the status of the selected patient is shown in a zoomed-in patient display 210 (FIG. 18) or alternative-embodiment zoomed-in patient display 220 (FIG. 19). In various embodiments the zoomed-in display shows a view of ALI/ARDS development (and/or development of another monitored condition), in time, for an individual patient. Optionally, the zoomed-in display may show predicted development in a given number of hours in the future. An ALI status indicator may be displayed as a value (optionally quantized) and corresponding color for all organ health assessment scores used in the ICUs (e.g. SOFA, AKIN criteria, et cetera, other contemplated scores including by way of illustrative example quantized CDS indicators for ALI, AKI, et cetera) in one concise, easy to read “snapshot” display. Trend indicators may be shown in various formats, such as using+/−signs, or up, down, horizontal arrows, by various color coding schemes (solid: traffic light pattern; spectrum-like: heatmap pattern; or so forth), by positive/negative numerical values, increased/decreased position on a vertical axis, or so forth. The combination of the overview display and the patient-specific zoom-in display provides a quick and easy mechanism for changing views/interfaces for groups of patients or individual patients and enables focusing on ALI or another organ system or syndrome of interest.

It is contemplated to enable customization of patient groups, organs/syndromes of interest, or scores used to represent a particular organ's health (e.g. RIFLE vs. AKIN criteria vs. CDS AKI indicator). Optionally, CDSS capability is incorporated to aid in decision making via display of suggested/recommended algorithm decision thresholds and in other embodiments, confidence intervals or bounds on this decision threshold.

In embodiments employing aggregation as previously described with reference to FIGS. 8 and 9, the zoomed-in view optionally shows results of constituent algorithms of the aggregation, optionally trended in time, that contribute to the aggregated algorithm output. While rectangular diagrammatic boxes are illustrated, markers used for organ health status can be of other shapes and of various sizes (e.g. actual traffic light, speedometer, or organ shape/image that changes color).

Current and recent past organ health information may be visualized via functionality including (by way of illustrative example): plotting; re-plotting from different starting points; animated plotting; pausing/resuming simulations; zooming (e.g. one-hour trends instead of six-hour trends); and so forth. In some embodiments, age of information, new or (carried) zero order held values, can be depicted via mechanisms such as filled/unfilled markers, outlined/not outlined markers, bolded/not bolded marker outlines, and so forth.

Without limiting the foregoing, the illustrative examples of FIGS. 16-19 are described in further detail in the following.

With reference to FIG. 16, a group overview display 200 is shown for an MICU including ten beds all occupied by patients. If a bed is empty, the text might say “Bed Empty”, the color might be light gray or faded, the action functions of the block are disabled, etc. If a bed is occupied, the block is labeled with a patient identifier (e.g. PID 123456). The text also includes a label and numeric value for the score of the organ indicator (e.g. ALI indicator SOFALI indicating severity of the ALI). Green, yellow, and red indicate low, medium, and high risk of ALI, respectively. In other embodiments, the color can be a spectrum of colors from lighter to darker hues. In still other embodiments, the color and score may indicate an overall organ health (e.g. respiratory, cardiovascular, renal, etc.). In still yet other embodiments, the scores for other organs can also be depicted. When multiple conditions are to be color-coded, the block is optionally segmented or has several components for each organ system, where each has the respective color and score indicating that organ's health.

With reference to FIG. 17, the overview display 200 of FIG. 16 is interacted with by a nurse to select another ICU (e.g. medical, surgical, trauma, etc.) via the drop-down GUI dialog 202. Instead of representing a specific ICU, additional groups of patients might include Worst10 (e.g. display the 10 most critically ill patients in all ICU's of the hospital or other medical center). User groups and number of beds (thus patients displayed) are as appropriate for the given ICU, and may be configurable for example using a “drag-and-drop” user interface by which a user drags a new bed into the ICU display and links it with a set of input data streams for that bed. (Similarly a bed can be removed by dragging it off the display).

In a contemplated variant embodiment of the overview display (not shown), the color coding conveys different information, namely being used to identify changes in parameters. For example, if a patient's organ status is declining, this can be reflected by “red” color coding even if the actual level of the ALI or other organ status indicator is not indicating ALI positive in this embodiment the color coding highlights changes rather than absolute values of organ status indicators.

With reference to FIG. 18, a zoomed-in display 210 is shown, which is suitably generated by the nurse selecting (e.g. clicking or double-clicking with a mouse, or touching in the case of a touchscreen) one diagrammatic box of the overview display 200 of FIG. 16 to select an individual patient to which to zoom. The illustrative patient of FIG. 18 has a high risk of ALI. Demographics are displayed in the upper right of the display 210. Demographics include but are not limited to height, weight, age, gender, predicted body weight, body mass index (BMI), hospital or ICU admission or discharge dates and times, chronic conditions, reasons for admissions, current diagnoses, and so forth. The upper left plot of the display 210 shows current and predicted ALI CDS algorithm output (aggregate SOFALI score on vertical axis, time on horizontal axis). The six lower left plots of the display 210 respectively plot each of the six individual algorithms that are aggregated to obtain the SOFALI score (cf. FIG. 9). For the plots in the lower left of each of the individual algorithms and the aggregated plot in the upper left, the recommended decision threshold (and optionally its confidence bounds) are optionally displayed as a line of value y on the vertical axis that spans the horizontal axis. The nurse or other user can select to review a new patient by using the drop down GUI dialog box in the uppermost left. The lower right side of the display shows a matrix of organ system health (SOFALI, cardiovascular, respiratory, renal, hepatic, coagulation) via colored markers over time (different colors are diagrammatically indicated in FIG. 18 by different shading levels). Markers could be different sizes, shapes, or images, can have bolded/non-bolded outlines to distinguish new values from old or carried values, and/or can increase or decrease in position on the vertical axis to represent increases and decreases in scores. Other embodiments could incorporate other clinical assessments (SOFA, AKIN, SIRS, etc.) or newly developed CDS assessments (CDS for ALI, AKI, DIC, etc.) or a combination of both. Selection of scores to be used or displayed is optionally customizable in a selectable preferences, configuration, or set-up window (not shown). In other embodiments, the focus organ system or the left side of the display can be changed to other organ systems by selecting a new organ to display. In other embodiments, a group or patient group (similar to or some version of figures above) may be displayed in the place of the individual algorithms. In some embodiments the nurse or other use can press a play button to animate plots and review patient health trends and trajectories over time from the start time or a selected time to the current time. Optional pause/resume functionality allows further analysis of particular points of concern. User interfacing for such controls is suitably implemented by user-controllable time slider bars or the like.

With reference to FIG. 19, an alternative embodiment zoomed-in display 220 is shown, in which the matrix of organ system health in the lower right side of the display is modified to employ a grid with numeric values in the grid cells. The organ system overview on the right side of the GUI includes the color-coding system as previously described (traffic light or spectrum-like, again diagrammatically represented in FIG. 19 by different shading levels). The color represents the current score, though other embodiments may include a numeric value for the current score as well. The “+/−” signs indicate a positive or negative trend from the previous value, where the higher or more positive the SOFA and SOFALI value, the worse the organ health. The numeric value immediately following a “+/−” sign is the delta or change from the previous value. Future embodiments can incorporate combinations of these current values and delta values or can use directional arrows instead of “+/−” signs.

With returning reference to FIG. 1, the disclosed techniques for detecting ALI or other conditions of concern for ICU patients are suitably implemented by the built-in computer, microprocessor, or so forth of the illustrative bedside monitor 10 and/or of the illustrative nurses' station electronic monitoring device 20. It will also be appreciated that the disclosed techniques can be embodied by a non-transitory storage medium storing instructions executable by such an electronic data processing device to perform the disclosed detection methods. The non-transitory storage medium may, for example, comprise a hard disk or other magnetic storage medium, random access memory (RAM), read-only memory (ROM), or another electronic storage medium, an optical disk or other optical storage medium, a combination of the foregoing, or so forth.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A non-transitory storage medium storing instructions executable by an electronic data processing device including a display to monitor a patient for acute lung injury (ALI) by operations including:
(i) receiving values of a plurality of physiological parameters for the patient;
(ii) receiving drug administration information pertaining to administration of one or more drugs to the patient;
(iii) computing an ALI indicator value based at least on the received values of the plurality of physiological parameters for the patient and the received drug administration information; and
(iv) displaying a representation of the computed ALI indicator value on the display.

2. (canceled)

3. (canceled)

4. The non-transitory storage medium of claim 1 wherein:
the receiving comprises receiving a data stream of values for the patient for each physiological parameter of the plurality of physiological parameters,
the computing comprises computing the ALI indicator value as a function of time based on the received data streams of values for the patient, and
the displaying comprises displaying a trend line representing the computed ALI indicator value as a function of time.

5. (canceled)

6. (canceled)

7. (canceled)

8. The non-transitory storage medium of claim 1 wherein:
the receiving comprises receiving a data stream of values for the patient for each physiological parameter of the plurality of physiological parameters, and
the computing comprises (1) computing a Lempel-Ziv complexity metric for each received data stream of values for the patient and (2) computing an aggregation of the Lempel-Ziv complexity metrics, the ALI indicator value being based at least on the aggregation of the Lempel-Ziv complexity metrics.

9. The non-transitory storage medium of claim 1 wherein the computing comprises:
computing the ALI indicator value based at least in part on applying a logistic regression model to the received values of the plurality of physiological parameters for the patient.

10. The non-transitory storage medium of claim 1 wherein the computing comprises:
computing the ALI indicator value based at least in part on applying a log-likelihood ratio (LLR) model to the received values of the plurality of physiological parameters for the patient.

11. The non-transitory storage medium of claim 1 wherein the computing comprises:
computing the ALI indicator value based at least in part on applying a trained model to the received values of the plurality of physiological parameters for the patient, the trained model having one or more model parameters trained on a training set comprising reference patients to distinguish between reference patients labeled ALI-positive and ALI-negative.

12. The non-transitory storage medium of claim 11 wherein the trained model comprises a Lempel-Ziv complexity metric model and the parameters include a threshold.

13. The non-transitory storage medium of claim 11 wherein the trained model comprises a logistic regression model and the parameters include coefficients βᵢ scaling respective received values xᵢ of the plurality of physiological parameters for the patient in the logistic regression model.

14. The non-transitory storage medium of claim 11 wherein the trained model comprises a log-likelihood ratio (LLR) model and the parameters include joint probabilities of received values dᵢ of the plurality of physiological parameters given ALI-positive and joint probabilities of received values dᵢ given ALI-negative.

15. The non-transitory storage medium of claim 1 wherein the computing comprises:
computing algorithm ALI indicator values for a plurality of different inference algorithms trained to discriminate between ALI-positive and ALI-negative patients; and
computing the ALI indicator value as an aggregation of the algorithm ALI indicator values.

16. The non-transitory storage medium of claim 15 wherein the computing of the ALI indicator value as an aggregation of the algorithm ALI indicator values comprises:
computing the ALI indicator value by applying linear discriminant analysis (LDA) to the algorithm ALI indicator values.

17. The non-transitory storage medium of claim 15 wherein the computing of the ALI indicator value as an aggregation of the algorithm ALI indicator values comprises:
computing the ALI indicator value by applying a voting analysis to the algorithm ALI indicator values.

18. The non-transitory storage medium of claim 1 further storing instructions executable by the electronic data processing device including the display to monitor a plurality of patients in an Intensive Care Unit (ICU) for ALI by operations including:
performing the operations (i) and (ii) for each patient to generate an ALI indicator value for each patient;
wherein the displaying operation (iii) comprises simultaneously displaying on the display a diagrammatic representation of each patient, the diagrammatic representation of each patient including an identification of the patient and a representation of the ALI indicator value for the patient.

19. (canceled)

20. An apparatus comprising:
an electronic data processing device including a display; and
a non-transitory storage medium as set forth in claim 1 operatively connected with the electronic data processing device to execute the instructions stored on the non-transitory storage medium to monitor a patient for acute lung injury (ALI).

21. (canceled)

22. (canceled)

23. A method comprising:
receiving values of a plurality of physiological parameters for a patient in an intensive care unit (ICU) at an electronic data processing device including a display;
receiving drug administration information pertaining to administration of one or more drugs to the patient;
using the electronic data processing device, computing an ALI indicator value (54, 78, 84) based at least on the received values of the plurality of physiological parameters for the patient and the received drug administration information using an inference algorithm trained on a training set comprising reference patients to distinguish between reference patients having ALI and reference patients not having ALI; and
displaying a representation of the computed indicator value on the display of the electronic data processing device.

24. (canceled)

25. (canceled)

26. (canceled)

27. (canceled)

28. (canceled)

29. (canceled)

30. (canceled)

31. (canceled)
